(19) United States
(12) Patent Application Publication
(10) Pub. No.: US 2023/0009363 A1
(43) Pub. Date: Jan. 12, 2023

(54) Determination System, Verification Device, and Determination Method

(71) Applicant: Nippon Telegraph and Telephone Corporation, Tokyo (JP)

(72) Inventors: Yurina Tanaka, Tokyo (JP); Soichi Oka, Tokyo (JP); Takashi Sakamoto, Tokyo (JP); Yuichi Akage, Tokyo (JP)

(21) Appl. No.: 17/785,237

(22) PCT Filed: Dec. 20, 2019
(86) PCT No.: PCT/JP2019/050145

§ 371 Date: Jun. 14, 2022

(51) Int. Cl. G16C 20/20; G16C 20/90

(52) U.S. Cl. CPC G16C 20/20; G16C 20/90

(57) ABSTRACT

Provided are a first measurement apparatus that measures the stable isotope ratios of a carbon element and a hydrogen element contained in hair of animal, and a verification device having a database including first reference data for the stable isotope ratios of the carbon element and the hydrogen element, which are classified for each production area of the hair of the animal, in which the first measurement apparatus measures the stable isotope ratios of the carbon element and the hydrogen element contained in the hair of the animal to be measured, and the verification device compares the measurement result of the first measurement apparatus with the first reference data, and determines the production area of the hair of the animal to be measured based on the comparison result.

### CROSS-REFERENCE TO RELATED APPLICATIONS

This application is a national phase entry of PCT Application No. PCT/JP2019/050145, filed on Dec. 20, 2019, which application is hereby incorporated herein by reference.

### TECHNICAL FIELD

The present invention relates to a determination system for determining a production area of hair of an animal.

### BACKGROUND

Cashmere is animal hair collected from cashmere goats, and the geographic regions where cashmere goats are bred are distributed from Central Asia to China, the Inner Mongolia Autonomous Region, and the like. Goats have long been used as livestock that can be raised even in harsh environments, but because they have a rough temperament and are less productive than cattle, pigs, and sheep, they are less likely to be raised as livestock and are mostly raised in geographic regions where it is difficult to raise other livestock.

Although it is said that the world production amount of cashmere (the production amount provided worldwide in one year) is about 20,000 to 30,000 tons, since it is a high-grade textile material, clothing products disguised as cashmere are also mixed in the distribution, four to five times as much cashmere has been provided to the market, and there has been demand to provide incontrovertible information on the production area of the raw materials.

When a manufacturer of fabric (knitted fabric) made from cashmere raw materials delivers products (fabric) to a clothing manufacturer, conventionally, the origin of the material was verified and certified by checking the transaction slips with dairy farmers, transaction slips with manufacturers who wash raw wool and sort only cashmere, transaction slips with yarn manufacturers, the diameter measurement (fineness measurement) and color of the hair using an optical microscope, and the like (see FIG. 6).

However, in addition to the time-consuming work of checking the consistency between the shipping record of the shipping source and the transaction slips and complicated slip management being necessary, the risk of counterfeit products being distributed has also been included. Moreover, consumers who use cashmere products had no choice but to trust the labeling on clothing tags.

In general, it is difficult to specify the production area of naturally-derived raw materials, and thus there was an inherent need to scientifically prove the production area of the raw materials. Conventionally, as a method of scientifically proving the production area of the raw materials, a method of using stable isotope ratios of hydrogen and oxygen contained in water molecules, which reflects the geographical difference of the production area, such as the latitude and altitude, has been known (e.g., see PTL 1 and NPL 1).

It is also known that naturally-derived raw materials and artificial materials have different stable isotope ratios of carbon that is contained therein. For example, it is known that products made by mixing artificial sugar into naturally-derived honey are on the market, and with food products such as honey, it is generally difficult to estimate the composition of raw materials by looking at the product itself, and therefore stable isotope ratio measurement of carbon has been used to evaluate the validity of the raw material of the product.

### CITATION LIST

### Patent Literature

[PLT 1] JP 2018-100834A

[Non-Patent Literature]

[NPL 1] Tsuguhiro Ohashi, Kenichiro So, Akihiro Yoshida, Hisashi Uematsu, Nobuaki Mochizuki, Takako Yasui, Ryoko Yoshimura, and Yoshihisa Sakai: “Research and Development Activities Related to Agriculture” NTT Technical Review, Vol. 14, No. 6, Jun. 2016.

### SUMMARY

### Technical Problem

As described above, consumers rely only on information provided by retailers for cashmere products, and there has been no way to verify the authenticity of the raw material production area written on the tag of a cashmere product, and the like. For retailers and producers in each step, it is possible to guarantee that transaction records before and after the step and the handling of products are proven, but since there is no proactive involvement in proving the validity of the transaction of the manufacturer and the handling of the product in a step prior to or subsequent to the step, it has been difficult to prove the production area of raw materials for cashmere products.

### Means for Solving the Problem

In order to achieve such an object, the determination system of embodiments of the present invention includes: a first measurement apparatus configured to measure stable isotope ratios of a carbon element and a hydrogen element contained in hair of an animal; and a verification device having a database including first reference data for the stable isotope ratios of the carbon element and the hydrogen element, which are classified for each production area of the hair of the animal, in which the first measurement apparatus measures stable isotope ratios of a carbon element and a hydrogen element contained in hair of an animal to be measured, and the verification device compares the measurement result of the first measurement apparatus with the first reference data and determines the production area of the hair of the animal to be measured based on the comparison result.

In order to achieve such an object, the verification device of embodiments of the present invention includes a database including first reference data for stable isotope ratios of a carbon element and a hydrogen element, which are classified for each production area of hair of an animal, in which a result of measuring stable isotope ratios of a carbon element and a hydrogen element contained in hair of an animal to be measured and the first reference data are compared, and the production area of the hair of the animal is determined based on the comparison result.

In order to achieve such an object, the determination method of embodiments of the present invention is a determination method to be performed in a determination system including a first measurement apparatus configured to measure stable isotope ratios of a carbon element and a hydrogen element contained in hair of an animal, and a verification device having a database including first reference data for the stable isotope ratios of the carbon element and the hydrogen element, which are classified for each production area of the hair of the animal, the method including: a step of, in the first measurement apparatus, measuring stable isotope ratios of the carbon element and the hydrogen element contained in hair of an animal to be measured, and a step of, in the verification device, comparing the result of measuring the stable isotope ratios of the carbon element and the hydrogen element contained in the hair of the animal to be measured and the first reference data, and determining the production area of the hair of the animal based on the comparison result.

### Effects of embodiments of the Invention

In embodiments of the invention of the present application, data obtained by evaluating and analyzing the scientific characteristics of cashmere itself, which is a raw material, is used in order to determine and authenticate whether the raw material information of various products is in line with the production area information. As a result, consumers will be able to identify the production area of raw materials for clothing products on their own, and retailers and producers in each step can eliminate or reduce slip management and slip comparison work, as well as provide a system that helps to guarantee a sense of security at the time of a commercial transaction and prevent forgery of the production area.

### BRIEF DESCRIPTION OF THE DRAWINGS

FIG. 1 is a diagram for describing a determination system according to the present embodiment.

FIG. 2A is a diagram for describing construction of a database in the determination system of the present embodiment.

FIG. 2B is an example of a configuration of a database according to the present embodiment.

FIG. 2C is a two-dimensional graph of reference data of a stable hydrogen isotope ratio and a stable carbon isotope ratio stored in the database of the present embodiment.

FIG. 3A is an example of one configuration of a tag of a clothing product according to the present embodiment.

FIG. 3B is an example of another configuration of a tag of a clothing product according to the present embodiment.

FIG. 3C is an example of another configuration of a tag of a clothing product according to the present embodiment.

FIG. 4A is a configuration example of a verification device according to the present embodiment.

FIG. 4B is an example of a configuration of a computer included in the verification device according to the present embodiment.

FIG. 5A is an example of a sequence for determining a production area or the like in the present embodiment.

FIG. 5B is an example of a flow for determining a production area or the like in the present embodiment.

FIG. 6 is a diagram for illustrating conventional material production area proof and authentication.

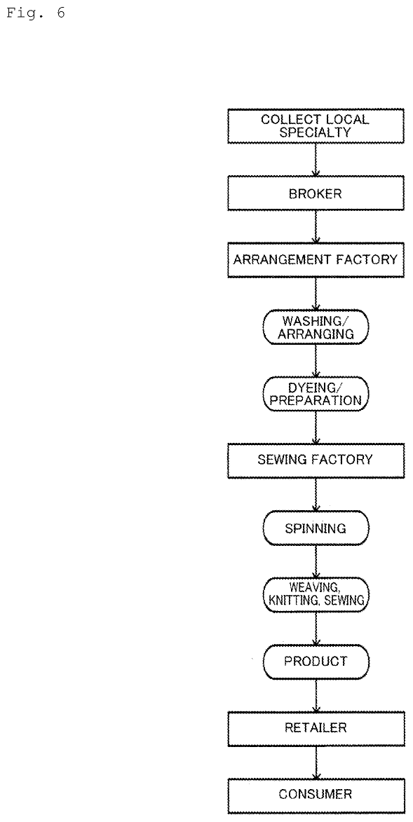

### DETAILED DESCRIPTION OF ILLUSTRATIVE EMBODIMENTS

Embodiments of the present invention will be described with reference to the drawings. Embodiments of the present invention can be implemented in various embodiments and is not limited to the embodiments described below.

Overview of embodiments of the Present Invention

In embodiments of the present invention, the stable isotope ratio of hydrogen contained in hair of an animal, which reflects the influence of the environment in which animals such as livestock are raised, and the stable isotope ratio of carbon contained in animal hair, which reflects the difference in feed for each breeder, are used. The determination system uses the relationship between the stable isotope ratio of hydrogen and the stable isotope ratio of carbon contained in animal hair, which depends on the production area stored in a database in advance, as an index for determining the production area, and responds with the validity of the original production area labeling to the user by comparing the results of measuring the stable isotope ratios of hydrogen and carbon in hair collected from the tag of the target product to be measured, with the index in the database.

Overview of Determination System

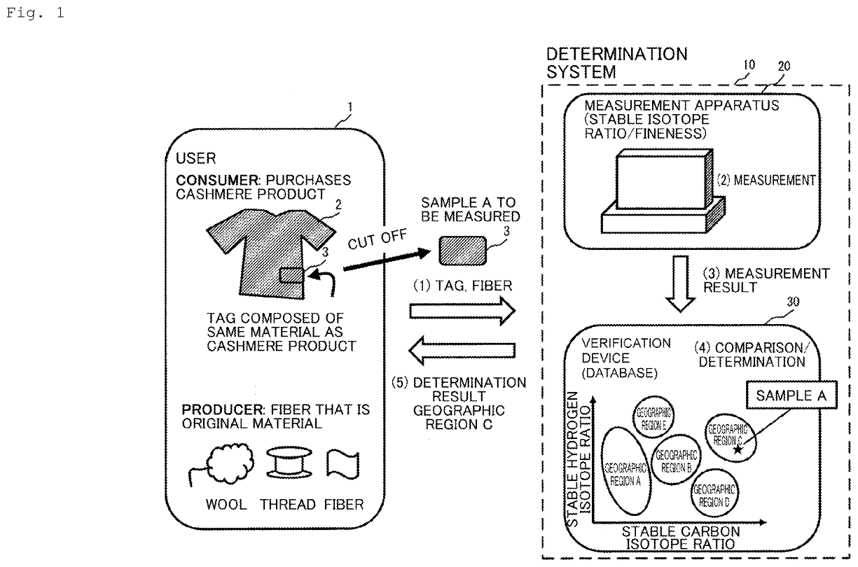

FIG. 1 is a diagram for illustrating an overview of the determination system of the present embodiment. The determination system 10 of the present embodiment is for use by a user 1 such as a consumer, a retailer, or a producer (a dairy farmer, a textile product producer, a fabric product producer, a clothing product producer). The determination system 10 is constituted by a measurement apparatus 20 that measures scientific analysis data that serves as a reference for determining the production area of hair of an animal provided by the user 1 and a verification device 30 that compares the measurement result with a database containing reference data. The hair of the animal to be measured is collected from a tag 3 attached to the clothing product 2 to be measured. The measurement apparatus 20 can be constituted by a plurality of apparatuses such as an apparatus (first measurement apparatus) for measuring the stable isotope ratio of an element contained in the hair of the animal according to the scientific analysis data to be measured and an optical microscope (second measurement apparatus) for measuring the fineness of hair of an animal.

In the present embodiment, the stable isotope ratio of hydrogen, which reflects the influence of the environment in which cashmere goats are raised, and the stable isotope ratio of carbon, which reflects the difference in feed of each breeder, are also used to determine the production area of animal hair. In the present embodiment, the reference data serving as an index for proving the production area, which consists of the relationship between the stable isotope ratio of hydrogen and the stable isotope ratio of carbon for each geographic region, and the measurement result of the hair collected from the tag of the clothing product are compared to provide the production area determination result. In the example of FIG. 1, the production area of sample A is determined to be geographic region C.

The following will describe a step of constructing a database including reference data for when proving the production area of cashmere, which is the basis of the present system, and the verification device and comparison step for comparing the measurement result with the tag attached to the product to be measured and the database.

Construction of Database

The following three measurement results are used in the step of constructing the database. The first is the result of measuring the stable isotope ratio of hydrogen as a geographic estimation of where the cashmere goat grew up, the second is the result of measuring the stable isotope ratio of carbon as a characteristic of the dietary components of the cashmere goat, and the third is the result of measuring the fineness with an optical microscope as an index of the degree of growth.

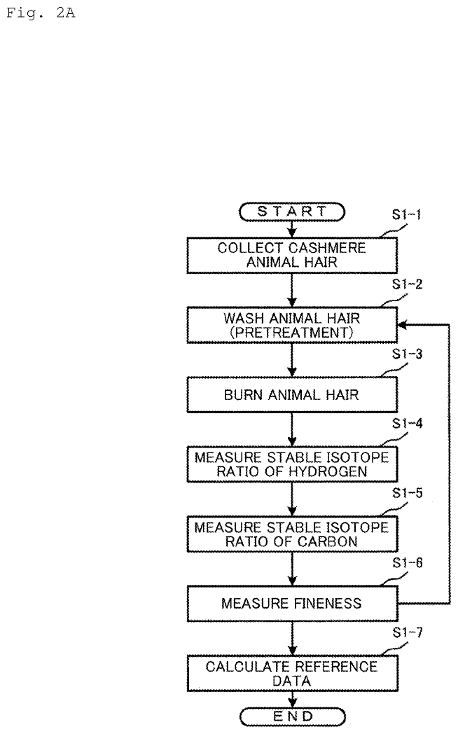

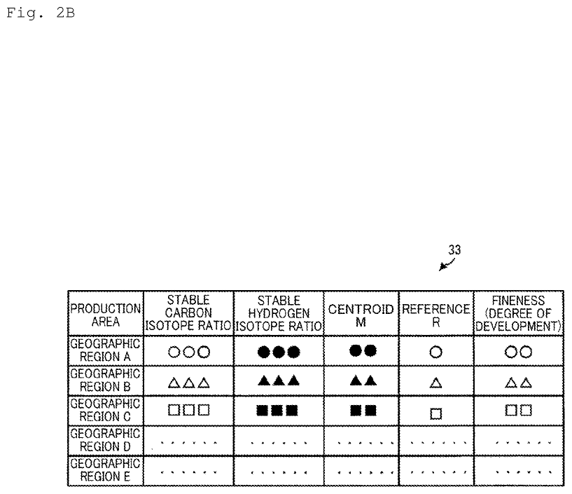

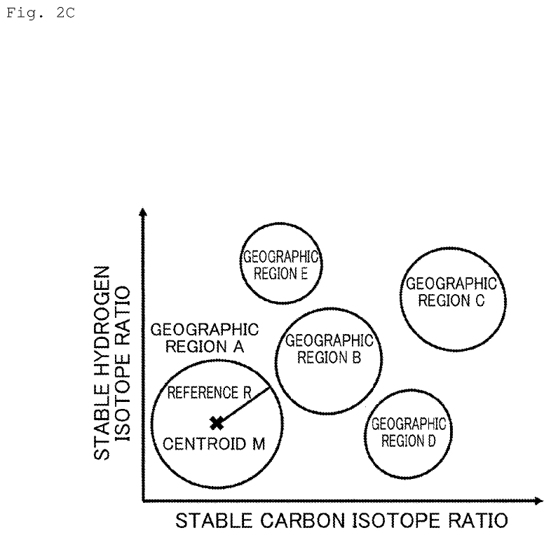

The step of constructing the database using FIGS. 2A to 2C will be described. First, cashmere goat hair may be purchased directly from a dairy farmer, and here, cashmere goat hair whose production area is certain may be purchased (S1-1).

In order to obtain information on only the animal hair at the time of measurement, contamination such as sand and dust adhering to the cashmere animal hair is removed from the purchased cashmere animal hair and freeze-drying is performed, and thereby pretreatment is performed such that only the animal hair component can be measured (S1-2).

The cashmere animal hair subjected to pretreatment is burned to generate a gas derived from the animal hair (S1-3), and the gas is used to perform stable isotope ratio measurement using a stable isotope ratio measurement apparatus (e.g., a laser absorption spectrometer, a stable isotope ratio mass spectrometer, a nuclear magnetic resonance spectrometer, etc.) (S1-4, S1-5).

First, stable hydrogen isotope ratio measurement is performed for geographic estimation (S1-4). In general, it is known that hydrogen contained in rain and the like has a different stable isotope ratio of hydrogen for each type of topography (for each latitude). Since the water intake differs depending on the habitat of cashmere goats, the habitat can be classified by measuring the stable isotope ratio of hydrogen.

Next, the stable isotope ratio of carbon, which reflects the dietary components, is measured (S1-5). The plants that cashmere goats ingest as food also differ depending on the production area. The stable isotope ratio of carbon reflects this difference. By measuring the stable isotope ratio of hydrogen and the stable isotope ratio of carbon, it is possible to estimate the production area of animal hair.

Furthermore, the fineness of cashmere animal hair may be measured using an optical microscope or the like (S1-6). Cashmere is known as high-grade animal hair, but baby cashmere made from the hair of baby cashmere goats is treated as even higher-grade animal hair. It is difficult to distinguish between a parent and a child by only measuring the isotope ratios of carbon and hydrogen. It is also possible to determine the validity of the degree of growth provided by the user by measuring the fineness, which is the diameter of the animal hair, storing the fineness, which indicates the degree of growth of the cashmere goat, in the database as reference data, and comparing the reference data that is the index of the degree of growth stored in the database and the fineness of the animal hair provided by the user.

At the time of the above-described measurement, it is possible to start with the measurement of the stable hydrogen isotope ratio or the measurement of the stable carbon isotope ratio, and any device that can perform measurement simultaneously may be used. Even with the same individual goat, it is desirable to carry out multiple measurements in consideration of variation in the stable isotope ratio at the cashmere collection sites (near the back and near the abdomen) and the possibility of error differences occurring in each measurement. It is also possible to improve the database by measuring multiple individuals in the same production area, assuming individual differences.

Finally, using the result of repeatedly measuring the same individual or multiple individuals, a numerical value that serves as a reference for data comparison is calculated (S1-7). As described above, it is known that in the stable isotope ratio measurement, even cashmere goats from the same production area take a plurality of measurement values within a certain range. For this reason, in order to use this measurement value as an evaluation index, the reference data is calculated using the following procedure.

Since the range of values that can be obtained by measuring the stable isotope ratio of hydrogen and the range of values that can be obtained by measuring the stable isotope ratio of carbon differ by about one order of magnitude, the numerical values are standardized in each range. Next, the centroid of each is calculated based on the result of measuring the stable isotope ratio of hydrogen and the stable isotope ratio of carbon a plurality of times (e.g., 10 times or more).

Calculation is performed using, as a reference, the Euclidean distance between the centroid and the measured value farthest from the centroid of the stable isotope ratio of hydrogen and the stable isotope ratio of carbon. As described above, a database including reference data that serves as an index for determining the production area is constructed.

FIG. 2B is an example of a configuration of the database. The database includes reference data (first reference data) for stable isotope ratios of carbon and hydrogen elements, which are classified for each animal hair production area, and reference data (second reference data) for fineness for determining the degree of growth of the animal. FIG. 2C is a two-dimensional graph of a Euclidean distance from the centroid of the two-dimensional data of the stable hydrogen isotope ratio and the stable carbon isotope ratio stored in the database. As a result of comparing the measurement result with the database, if the measurement result is within the range of the Euclidean distance from the centroid, it can be determined that the production area is the production area in that geographic region. Note that in this embodiment, the stable isotope ratios of hydrogen and carbon are used to determine the production area of animal hair, but the production area may also be determined by combining the stable isotope ratio of nitrogen, which is one of the main constituents of living organisms.

Tag for Determining Production Area

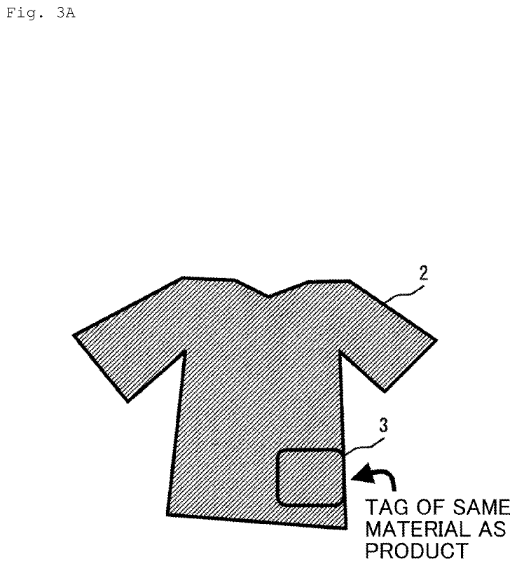

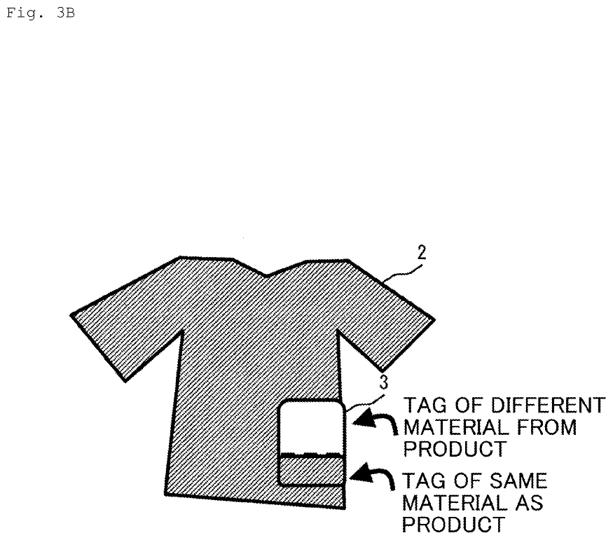

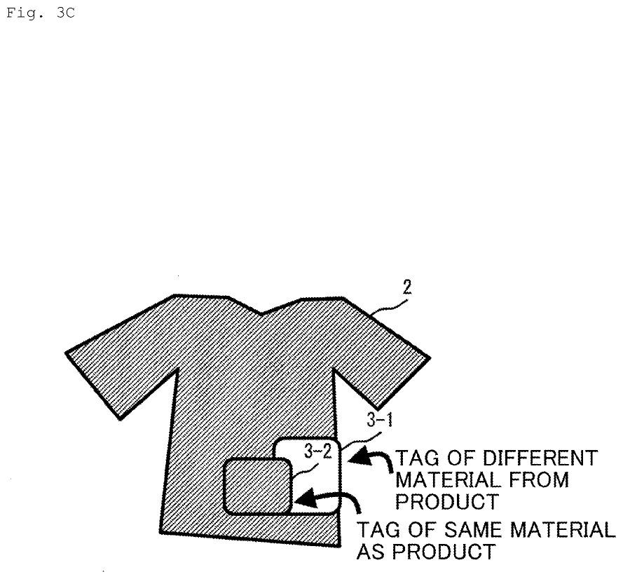

The tag attached to the clothing products to be measured will be described with reference to FIGS. 3A to 3C.

As described above, in order to perform a scientific analysis of the hair of an animal, it is necessary to burn cashmere, and therefore a portion of the actual cashmere product is required. At that time, assuming a case where the validity of the raw material labeling of a cashmere product such as a sweater is confirmed, if a portion of the sweater is cut out in order to carry out the proof of the production area, the sweater itself can no longer be used. It is desirable to have a system according to which it is possible to scientifically analyze cashmere products without damaging the cashmere products themselves, and provide the results of determination of the production area of the raw materials desired by the user.

In view of this, in the present embodiment, the production area is determined using the animal hair collected from the display label (tag) 3 sewn on the lining of the clothing product 2 or the like in order to determine the production area of the raw material of the product itself without affecting the clothing product itself.

For example, as the configuration of the tag 3, a configuration is conceivable in which the tag 3 itself, which is sewn on the clothing product, is made of the same fabric as the clothing product 2 (FIG. 3A), a portion of the tag 3 is made of the same fabric as the clothing product (FIG. 3B), and another tag (3-2) made of the same fabric as the clothing product 2 is sewn together with the tag (3-1) (FIG. 3C), or the like. By collecting the animal hair to be measured from the tag of the clothing product produced using the animal hair, it is possible to determine the raw material production area of the clothing product without affecting the clothing product itself.

Configuration of Verification device

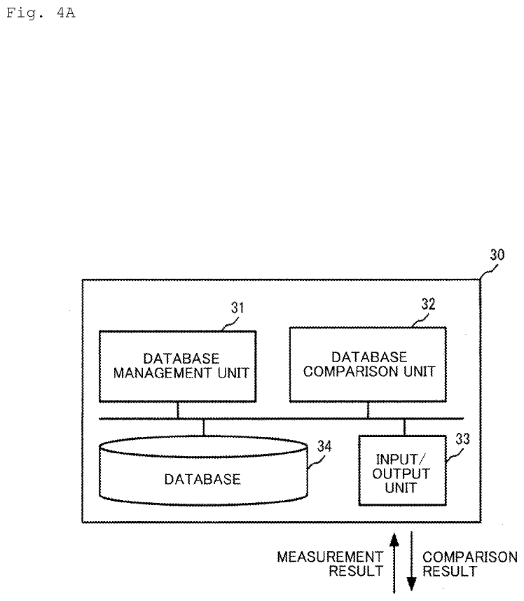

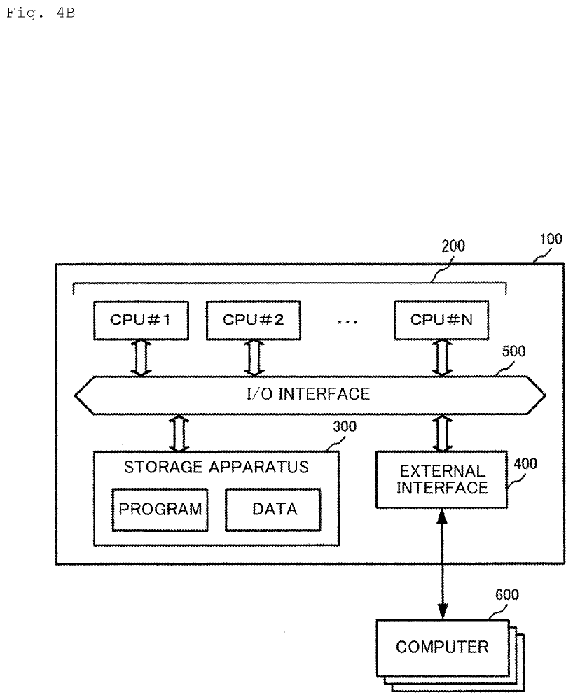

FIG. 4A is an example of a configuration of a verification device that compares the measurement result with the database.

The verification device 30 is constituted by a database management unit 31 that manages a database 34, a database comparison unit 32 that compares the measurement result with the database 34, an input/output unit 33 that transmits and receives the measurement result and the comparison result, and the database 34 in which reference data (first reference data) for stable isotope ratios of carbon elements and hydrogen elements classified for each animal hair production area and reference data (second reference data) for fineness for determining the degree of growth of the animals are recorded.

The database management unit 31, database comparison unit 32, input/output unit 33, and database 34 of the verification device 30 can be realized by, for example, a computer including a CPU (Central Processing Unit), a storage apparatus, and an external interface (hereinafter, external I/F), and a program that controls these hardware resources. An example of a configuration of such a computer is shown in FIG. 4B.

The computer 100 includes a CPU 200, a storage apparatus 300, and an external I/F 400, which are connected to each other via an I/O interface 500. The program for comparing the data of the present embodiment, the reference data for the stable isotope ratios of the carbon element and the hydrogen element, and the reference data for the fineness are stored in the storage apparatus 300, and another computer that receives input of the measurement data and outputs the comparison result is connected to the external I/F 400. The CPU 200 executes the processing described in the present embodiment according to a program or the like stored in the storage apparatus 300. Also, the processing program may be configured to be recorded on a computer-readable recording medium.

Comparison Between Measurement Result and Database

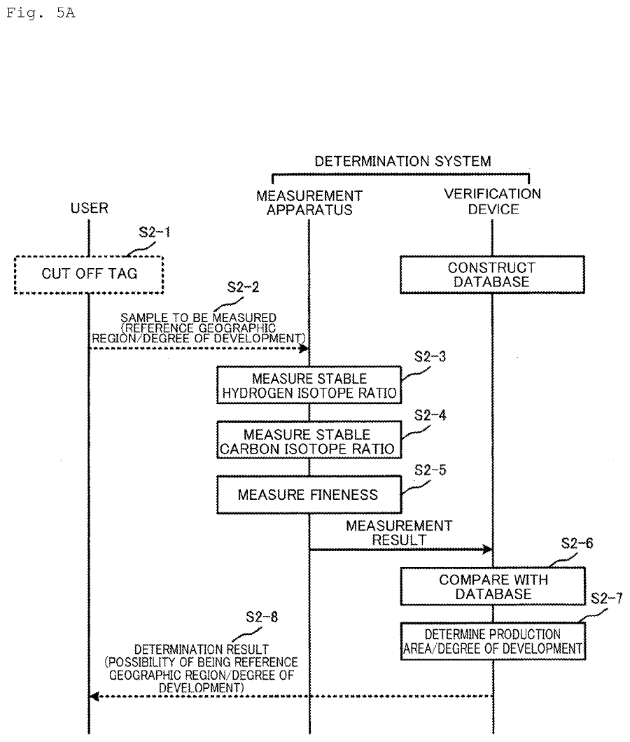

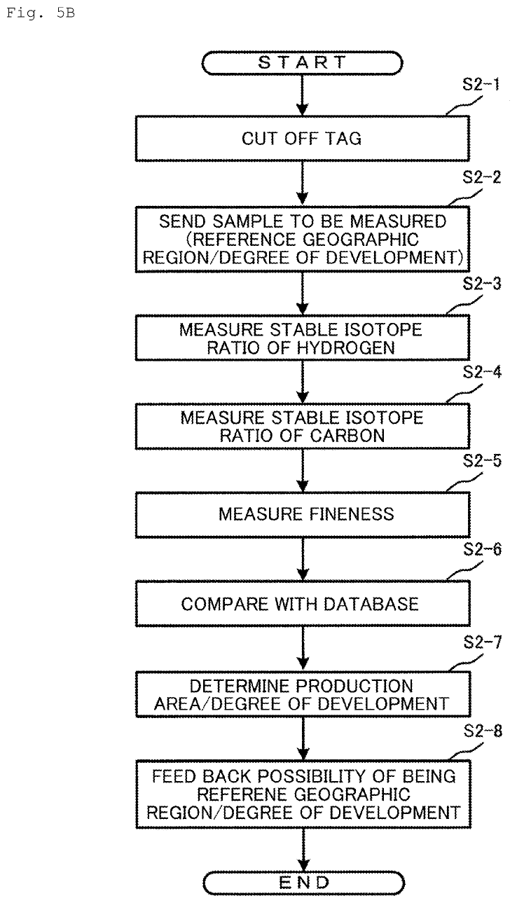

A step of comparing the measurement result and the database will be described with reference to FIGS. 5A and 5B. FIGS. 5A and 5B are examples of a sequence and a flow of a method for determining a production area and the like.

Only an amount of animal hair that is needed for measurement of animal hair that is the same as that of the clothing product to be measured is cut out as a measurement sample from the tag of the clothing product to be measured to determine the production area (S2-1).

The tag cut out from the clothing product is sent as a measurement sample to be measured to a determination system having a measurement apparatus such as a stable isotope ratio measuring device (S2-2). At this time, the geographic region to be referred to can also be specified as a reference geographic region.

For example, if the validity of the production area of cashmere purchased as being produced in Inner Mongolia is to be determined, it is also possible to send the information “Inner Mongolia” together with the measurement sample. Furthermore, it is also possible to send the reference degree of growth to be referred to together with the reference geographic region to be referred to. For example, in order to determine the validity of being baby cashmere, information indicating the degree of growth of animals for baby cashmere and the like can be sent.

The measurement apparatus of the determination system measures the stable isotope ratios of carbon and hydrogen and the fineness of the sent sample to be measured (S2-3 to S2-5) in the same manner as when constructing the database.

By comparing the measurement result and the database of the geographic region to be referred to, it is possible to quantify the possibility that the cashmere was produced in the reference geographic region (S2-6, S2-7). As the comparison result, the result of dividing the Euclidean distance calculated based on the measurement result and the centroid (M) of the reference geographic region, and the reference Euclidean distance (R) in the database is used. If the division result is 1 or less, it can be determined that the reference geographic region is valid. By feeding back the numerical value obtained as a result of the comparison to the user as the determination result, it is possible to provide a mechanism by which the user can confirm the validity of the production area. Also, by comparing the measured fineness and the fineness in the database, it is possible to provide the validity of the degree of growth, such as whether or not the cashmere is baby cashmere.

Also, tags such as the raw material labeling, the original production area labeling, and the brand mark have conventionally been used in clothing products, but by attaching tags made of the same material as the clothing product to the clothing product, it is possible to determine the production area and the degree of growth without affecting the clothing product itself.

Also, by providing a system like the one described in the present embodiment and fostering a sense of security for users and reliability for business partners, it is possible to increase the degree of recognition of cashmere products and contribute to the revitalization of the market. Furthermore, it will also be possible to provide a common cashmere inspection reference in the international market.

By using the system and service of the present embodiment, for example, in the market handling recycled clothing and the like, it is possible to realize the distribution of products for which the objective raw material production area obtained using scientific analysis results is guaranteed as one product quality guarantee.

Extension of Embodiment

Although the invention of the present application has been described above with reference to the embodiment, the invention of the present application is not limited to the above-described embodiment. Various changes that can be understood by those skilled in the art can be made to the configuration and details of the invention of the present application within the scope of the technical idea of the invention of the present application.

The database that serves as an index for determining the production area does not necessarily need to be constructed by a person who has a measurement apparatus, and a person who has a measurement apparatus may be requested to perform measurement, and another person may compare that measurement result with the database. Also, a person who has only a measurement apparatus may provide a determination service with an agreement with a person who has a database.

By providing a product with a tag made of the same material as the clothing product, for example, if the clothing has holes due to insect damage or getting caught, there is also a possibility that a non-conventional tag usage method, such as the tag being cut off and used as a patch for repair, will be provided.

The determination system may also be provided by a testing facility for textile products and materials and the like, and can also be provided by various companies and individuals as a service on an NW. For example, it is possible to develop an apparel service according to which it is possible to compare the production area in parallel with the sale of products at stores as well.

It is possible for an apparel manufacturer to manufacture and provide a tag using the same material as the product, and a retail store or the like may specify the attachment of the tag. For yarn products and fabric products as well, a tag composed of the same material may be provided along with the product or adhered to a box or the like in which the yarn product or fabric product is packaged.

### INDUSTRIAL APPLICABILITY

Embodiments of the present invention can be used in an authentication system for authenticating the production area described on the tag of a clothing product made of the hair of an animal.

### REFERENCE SIGNS LIST

1 User

2 Clothing product

3 Tag

10 Determination system

20 Measurement apparatus

30 Verification device.

1.-8. (canceled)

9. A determination system comprising:
a first measurement apparatus configured to measure stable isotope ratios of a carbon element and a hydrogen element contained in hair of an animal to be measured; and
a verification device having a database that includes first reference data for stable isotope ratios of the carbon element and the hydrogen element contained in hair of animals, the first reference data classified according to production areas of the hair of the animals, wherein the verification device is configured to:
compare the stable isotope ratios measured by the first measurement apparatus with the first reference data; and
determine the production area of the hair of the animal to be measured based on a comparison result between the stable isotope ratios measured by the first measurement apparatus and the first reference data.

10. The determination system according to claim 9, further comprising:
a second measurement apparatus configured to measure fineness of the hair of the animal to be measured;
wherein the database includes second reference data for fineness of the hair of animals;
the verification device is further configured to:
compare the fineness measured by the second measurement apparatus with the second reference data; and
determine a degree of growth of the animal to be measured based on the comparison result between the fineness measured by the second measurement apparatus and the second reference data.

11. The determination system according to claim 9,
wherein the first reference data includes data on a centroid of two-dimensional data of the stable isotope ratios of the carbon element and the hydrogen element of the hair of the animals and data on a Euclidean distance from the centroid.

12. The determination system according to claim 9,
wherein the hair of the animal to be measured is included in a clothing product produced using the hair of the animal and is collected from a tag including hair of the animal that is the same as that in the clothing product.

13. A verification device, comprising
a database including first reference data for stable isotope ratios of a carbon element and a hydrogen element contained in hair of animals, the first reference data classified according to production areas of hair of the animals, and
a processor configured to:
compare stable isotope ratios of the carbon element and the hydrogen element contained in hair of an animal to be measured with the first reference data; and
determine a production area of the hair of the animal to be measured based on a comparison result between the stable isotope ratios of the carbon element and the hydrogen element contained in the hair of the animal to be measured and the first reference data.

14. The verification device according to claim 13, wherein:
the database further includes second reference data for fineness of hair of animals; and
the processor is further configured to:
compare a fineness of hair of an animal to be measured and the second reference data; and
determine a degree of growth of the animal to be measured based on the comparison result between the fineness of the hair of the animal to be measured and the second reference data.

15. The verification device according to claim 13,
wherein the first reference data includes data on a centroid of two-dimensional data of the stable isotope ratios of the carbon element and the hydrogen element of the hair of the animals and data on a Euclidean distance from the centroid.

16. The verification device according to claim 13,
wherein the hair of the animal to be measured is included in a clothing product produced using the hair of the animal and is collected from a tag including hair of the animal that is the same as that in the clothing product.

17. A determination method to be performed in a determination system including a first measurement apparatus configured to measure stable isotope ratios of a carbon element and a hydrogen element contained in hair of an animal to be measured, and a verification device having a database that includes first reference data for stable isotope ratios of the carbon element and the hydrogen element contained in hair of animals, the first reference data classified by production areas of the hair of the animals, the method comprising:
a step of, performed by the first measurement apparatus, measuring stable isotope ratios of the carbon element and the hydrogen element contained in hair of an animal to be measured, and
a step of, performed by the verification device, comparing the stable isotope ratios of the carbon element and the hydrogen element contained in the hair of the animal to be measured with the first reference data, and determining a production area of the hair of the animal to be measured based on a comparison result between the stable isotope ratios of the carbon element and the hydrogen element contained in the hair of the animal to be measured and the first reference data.

18. The determination method according to claim 17, wherein the determination system further includes a second measurement apparatus configured to measure fineness of the hair of the animal to be measured, and the database further includes second reference data for the fineness of the hair of the animals.

19. The determination method according to claim 18, wherein the method further comprises:
a step of, performed by the second measurement apparatus, measuring the fineness of the hair of the animal to be measured; and
a step of, performed by the verification device, comparing the fineness of the hair of the animal to be measured with the second reference data and determining a degree of growth of the animal to be measured based on the comparison result between the fineness of the hair of the animal to be measured and the second reference data.

20. The determination method according to claim 17,
wherein the first reference data includes data on a centroid of two-dimensional data of the stable isotope ratios of the carbon element and the hydrogen element of the hair of the animals and data on a Euclidean distance from the centroid.

21. The determination method according to claim 17,
wherein the hair of the animal to be measured is included in a clothing product produced using the hair of the animal and is collected from a tag including hair of the animal that is the same as that in the clothing product.
